(19) United States
(12) Patent Application Publication
(10) Pub. No.: US 2026/0041430 A1
(43) Pub. Date: Feb. 12, 2026

(54) MULTI-COMPONENT VASCULAR OCCLUSION DEVICES

(71) Applicant: Boston Scientific Scimed, Inc., Maple Grove, MN (US)

(72) Inventors: Daniel K. Tomaschko, Savage, MN (US); Nicholas Lee Tassoni, Andover, MN (US)

(73) Assignee: Boston Scientific Scimed, Inc., Maple Grove, MN (US)

(21) Appl. No.: 19/286,678

(22) Filed: Jul. 31, 2025

Related U.S. Application Data
(60) Provisional application No. 63/679,752, filed on Aug. 6, 2024.

(51) Int. Cl. A61B 17/12

(52) U.S. Cl. CPC A61B 17/12109; A61B 17/12031; A61B 17/12145; A61B 17/12172; A61B 17/12186; A61B 2017/1205

(57) ABSTRACT

A vascular occlusion device for deployment within a lumen of a vessel may include an expandable anchor component configured to shift between a collapsed configuration and an expanded configuration, the expandable anchor component, where the expandable anchor component is configured in the expanded configuration to exert a radially outward force against an interior wall of the vessel; and an embolic component separate from and disposed at least partially within the expandable anchor component.

### CROSS-REFERENCE TO RELATED APPLICATIONS

This application claims the benefit of U.S. Provisional Patent Application Ser. No. 63/679,752 filed on Aug. 6, 2024, the disclosure of which is incorporated herein by reference.

### TECHNICAL FIELD

The present disclosure pertains to medical devices, kits, and methods for manufacturing, forming, or using multi-component vascular occlusive medical devices. More particularly, the present disclosure pertains to multi-component vascular occlusion devices.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

In some aspects, the present disclosure pertains to a multi-component vascular occlusion device for deployment within a lumen of a vessel, the multi-component vascular occlusion device comprising: an expandable anchor component configured to shift between a collapsed configuration and an expanded configuration, wherein the expandable anchor component is configured in the expanded configuration to exert a radially outward force against an interior wall of the vessel; and an embolic component separate from and disposed at least partially within the expandable anchor component.

In some aspects, which can be used in conjunction with any of the above aspects, wherein the embolic component is configured to not be disposed within the expandable anchor component when the expandable anchor component is initially in the collapsed configuration and subsequently be disposed at least partially within the expandable anchor component while the expandable anchor component is subsequently in the expanded configuration.

In some aspects, which can be used in conjunction with any of the above aspects, wherein the expandable anchor component comprises a frame including a wire, a mesh, or a screen.

In some aspects, which can be used in conjunction with any of the above aspects, wherein the expandable anchor component comprises a coil.

In some aspects, which can be used in conjunction with any of the above aspects, wherein the embolic component is a mechanical embolic component, the mechanical embolic component including a wire, a mesh, or a screen.

In some aspects, which can be used in conjunction with any of the above aspects, wherein the embolic component comprises an embolic fluid, the embolic fluid comprising a embolic liquid, embolic foam, or an embolic gel.

In some aspects, which can be used in conjunction with any of the above aspects, wherein the expandable anchor component is configured to be deployed within the lumen of the vessel, and wherein the embolic component is configured to be deployed within the lumen of the vessel subsequent to deployment of the expandable anchor component within the lumen of the vessel.

In some aspects, which can be used in conjunction with any of the above aspects, further comprising an entrapment component that is separate from the expandable anchor component and the embolic component.

In some aspects, which can be used in conjunction with any of the above aspects, the entrapment component is configured to be deployed within the lumen of the vessel subsequent to deployment of the expandable anchor component and prior to the embolic component within the lumen of the vessel.

In some aspects, which can be used in conjunction with any of the above aspects, wherein the expandable anchor component defines a perimeter of an interior space disposed within the expandable anchor component and the entrapment component is configured to: friction fit at least partially within the perimeter of the interior space disposed within the expandable anchor component; or abut a proximal end of the expandable anchor component while the expandable anchor component exerts the radially outward force against the interior wall of the vessel in the expanded configuration.

In some aspects, which can be used in conjunction with any of the above aspects, wherein the entrapment component includes a mesh, a coil, a liquid, a foam, a gel, or any combination thereof.

In some aspects, which can be used in conjunction with any of the above aspects, wherein the expandable anchor component is a coil.

In some aspects, which can be used in conjunction with any of the above aspects, wherein the embolic component is a liquid embolic material.

In some aspects, which can be used in conjunction with any of the above aspects, wherein the expandable anchor component includes a repositioning feature.

In some aspects, which can be used in conjunction with any of the above aspects, wherein at least the expandable anchor component and the embolic component are configured to be deployed via the same individual microcatheter.

In some aspects, the present disclosure pertains to a multi-component vascular occlusion device for deployment within a lumen of a vessel, the multi-component vascular occlusion device comprising: an expandable anchor component configured to shift between a collapsed configuration and an expanded configuration, the expandable anchor component defining a perimeter of an interior space disposed within the expandable anchor component, wherein the expandable anchor component is configured in the expanded configuration to exert a radially outward force against an interior wall of the vessel; an entrapment component separate from the expandable anchor component and including a mesh that is friction fit at least partially within the perimeter of the interior space disposed within the expandable anchor component or abuts a proximal end of the expandable anchor component; and an embolic component separate from the expandable anchor component and the entrapment component, wherein the embolic component is disposed i) at least partially within the mesh and ii) at least partially within the expandable anchor component to contact at least a portion of the perimeter of the interior space.

In some aspects, which can be used in conjunction with any of the above aspects, wherein the vessel has an inner diameter of about 6 millimeters or greater, and wherein the expandable anchor component is a coil configured to exert the radially outward force against the interior wall of the vessel in the expanded configuration that is sufficient to retain the multi-component vascular occlusion device in the vessel.

In some aspects, the present disclosure pertains to a kit for forming a multi-component vascular occlusion device for deployment within a lumen of a vessel, the kit comprising: an expandable anchor component configured to shift between a collapsed configuration and an expanded configuration, wherein the expandable anchor component is configured to exert a radially outward force against an interior wall of the vessel in the expanded configuration; and an embolic component separate from and configured to be disposed at least partially within the expandable anchor component while the expandable anchor component exerts the radially outward force against the interior wall of the vessel to form the multi-component vascular occlusion device.

In some aspects, which can be used in conjunction with any of the above aspects, wherein the expandable anchor component and the embolic component are separate elements.

In some aspects, which can be used in conjunction with any of the above aspects, wherein the kit further comprises an entrapment component, and wherein the entrapment component is separate from the expandable anchor component and the embolic component.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1A illustrates aspects of an example of an expandable anchor component disposed in a lumen of a vessel;

FIG. 1B illustrates an example of a multi-component vascular occlusion device including an embolic component and the expandable anchor component of FIG. 1A;

FIG. 2A illustrates aspects of another example of an expandable anchor component disposed in a lumen of a vessel;

FIG. 2B illustrates aspects of an example of an entrapment component disposed in the lumen of the vessel of FIG. 2A;

FIG. 2C illustrates another example of a multi-component vascular occlusion including an embolic component, the expandable anchor component of FIG. 2A, and the entrapment component of FIG. 2B;

FIG. 3 illustrates an example of a kit for forming an example of a multi-component vascular occlusion device;

FIG. 4 illustrates an example of a kit for forming another example of a multi-component vascular occlusion device;

FIG. 5 illustrates an example of a method of forming an example of a multi-component vascular occlusion device; and

FIG. 6 illustrates an example of a method of forming of another example of a multi-component vascular occlusion device.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean a greatest measurement of a stated or identified dimension, unless specifically referred to as a minimum extent. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. However, where referred to as a “minimum extent”, the “extent” shall refer to a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact and/or are affected by the cardiovascular system are prevalent in populations throughout the world. Vascular occlusion, or embolization, may be used to treat such diseases and/or medical conditions, including bleeds, aneurysms, or venous insufficiency, among others.

Embolization may be used to stop (e.g., occlude) blood flow in a variety of different sized vessels such as those having small (<1-millimeter (mm)) diameters and those having large diameters (e.g., diameters in a range from about 6 mm to about 10 mm). Examples of traditional devices intended to stop blood flow include those having one or more vascular plugs (e.g., one or more conformable balloons), an individual coil formed of a plurality of materials and/or different sized materials that may yield different mechanical properties (e.g., different stiffness or rigidity) along a longitudinal length of traditional devices, and/or that employ a plurality of coils with different mechanical properties. For instance, the traditional devices may be all-in-one devices having various elements that are permanently coupled together e.g., are coupled together at least prior to and during to deliver to a target site in a vessel. However, the traditional devices may be prone to various issues such as requiring large catheters for delivery and/or being difficult to position and retain at a target site (e.g., being prone to migration from a target site and/or being prone to elongation (e.g., an undesired degree of longitudinal elongation) during and/or subsequent to deployment at the target site). Such difficulties may be magnified when the traditional devices are deployed at a target site in a large diameter vessel (e.g., having an internal diameter of six millimeters or greater), for instance, due to the presence of a relatively large head pressure imparted by blood flow in the larger vessels.

Disclosed herein are multi-component medical devices, kits, and methods that may be used within a portion of the cardiovascular system to treat and/or repair some arterial venous malformations and/or other diseases or conditions, and methods of making such devices. The multi-component vascular occlusion devices, kits, and methods disclosed herein may also provide a number of additional desirable features and benefits as described herein. For instance, the multi-component vascular occlusion devices can provide improved deliverability to, positioning at, and/or retention at a target site in a blood vessel at least due in part to providing the multi-component medical devices herein as separate components rather than as an all-in-one device. For example, the multi-component vascular occlusion devices herein may permit the use of smaller delivery devices (e.g., microcatheters), may permit the use of an individual delivery catheter to deliver each of the separate components of the multi-component medical device to a target site, may be less prone to elongation and/or migration, and/or may permit the multi-component vascular occlusion devices to be readily positioned (e.g., repositioned) at a target site within a lumen of a vessel, may provide a larger radial force against an inner wall of a vessel, and in view of the above may permit the quick and long lasting occlusion of a vessel, as compared to traditional occlusion devices such as those described herein.

As used herein, having the components of the multi-component device be “separate” components refers to the components not being coupled together at least prior to and during delivery to a target site in a vessel. As such and unlike all-in-one devices, the individual components of the multi-component vascular occlusion devices can be delivered separately to a target site at which point the multi-component vascular occlusion device can be formed. For instance, the respective separate individual components may contact with each other once at the target site in a lumen of a vessel. For example, the individual components of the multi-component vascular occlusion devices herein can be delivered sequentially or consecutively to a target site by one or more insertion devices. At least due in part to employing separate individual components, the multi-component medical devices herein permit the use of smaller dimension delivery devices (e.g., 5 FR, 4 FR, or less catheters) because the individual components can have radial cross-sections that are smaller than corresponding radial cross-sections of all-in-one devices and/or can permit the use of an expandable anchor component that can provide a higher radial holding force, for instance, than comparative radial forces provided by traditional occlusion devices such as traditional all-in-one inclusion devices.

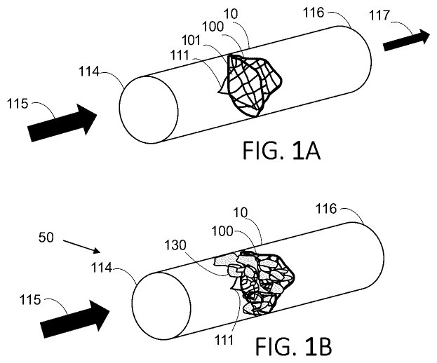

As illustrated in FIGS. 1A and 1B, in some embodiments, an example multi-component vascular occlusion device 50 for deployment within a lumen of a vessel 10 may comprise an expandable anchor component 100. The expandable anchor component 100 can be formed of one or more of the materials described herein. In some embodiments, the expandable anchor component 100 can be formed of a continuous individual material. The expandable anchor component can be formed of one or more wires or filaments. For example, the expandable anchor component 100 may be made from a shape memory material, that may be heat set to a predetermined configuration, or may have other characteristics capable of influencing its behavior. In some embodiments, the expandable anchor component 100 may be mechanically self-expandable or is balloon expandable. Other configurations are also contemplated.

The expandable anchor component 100 can function at least primarily to anchor the multi-component vascular occlusion devices herein at a target site in a vessel lumen. For instance, the expandable anchor component 100 can be configured to provide a radial holding force that is greater than an expected force or head pressure exerted on the multi-component vascular occlusion devices herein at a target site in a vessel lumen. In some embodiments, a holding force imparted by the expandable anchor component 100 can be tailored to a particular vessel (e.g., based on a location of a target site along the intended vessel and/or a diameter of the vessel) in which the multi-component vascular occlusion devices herein are to be deployed.

The expandable anchor component 100 may include a body formed from a plurality of interconnected coils or the body can be formed from struts, one or more wires or polymer segments/filaments, and/or a lattice support structure (e.g., forming a frame). In some embodiments, the body of the expandable anchor component 100 may be integrally formed. Stated differently, the body of the expandable anchor component 100 may be a unitary member such as a coil formed of a series of continuous sections of coils which are woven or otherwise formed into a frame. However, in some embodiments, the expandable anchor component 100 may be formed of various elements such as a plurality of coils and/or a plurality of wires or filaments which are coupled together. For example, the body of the expandable anchor component 100 may have the form and/or appearance of an expandable coil or an expandable stent or basket, as described herein. The body of the expandable anchor component 100 may define a perimeter of an interior space 101 disposed within the expandable anchor component 100.

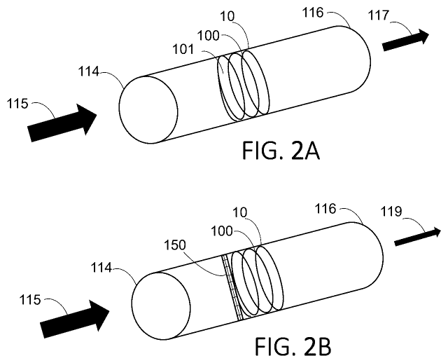

In some embodiments, the expandable anchor component 100 may comprise a frame, a coil, or a combination of a frame and a coil. The coil can be a metallic coil and/or polymeric coil. In some embodiments, the expandable anchor component 100 can be a coil as illustrated in FIG. 2A.

However, in some embodiments the expandable anchor component 100 can be a frame, as illustrated in FIGS. 1A-1B. The frame can be a metallic frame and/or a polymeric frame. The frame can include or be formed of a wire, a mesh, and/or a screen. The frame can be a polymeric frame formed from a shape-memory material. The frame can be a radially expandable basket or stent-like structure. For instance, the frame can be a radially expandable basket, for instance, which has a proximal end region with a larger radial cross-section than a radial cross-section of the distal end region of the basket, as illustrated in FIG. 1A. For example, the proximal end region of the frame may be configured with a radial cross-section sufficient to contact and/or anchor the frame to an inner wall of a vessel and the distal end region of the basket may have a smaller radial cross-section (e.g., which is configured to float within the vessel or not contact the inner wall of the vessel, as illustrated in FIG. 1A). In such instances, the frame can be tapered from the proximal end region to the distal end region. Employing frames which are tapered or otherwise have a proximal end region with a larger radial cross-section than a radial cross section of the distal end region can promote aspects herein such as promoting retention of the embolic component 130 at least partially within the frame (e.g., within interstitial spaces between adjacent frame wires, segments, or cells) and thereby promotes formation of multi-component vascular occlusion devices.

The expandable anchor component 100 may be configured to shift between a collapsed configuration and an expanded configuration upon delivery to a treatment site within the vessel 10. In the expanded configuration, the body of the expandable anchor component 100 may be “open” or enlarged compared to the collapsed configuration. In the expanded configuration, the body of the expandable anchor component 100 may define an outer diameter and/or outer extent greater than the outer diameter or outer extent of the body of the expandable anchor component 100 in the collapsed configuration.

In some embodiments, the expandable anchor component 100 may be configured to exert a radially outward force against an interior wall of the vessel 10 in the expanded configuration and/or as the expandable anchor component 100 approaches or is shifting toward the expanded configuration. The radially outward force against the interior wall of the vessel 10 may be configured to anchor the multi-component vascular occlusion devices herein, so as to prevent migration of any of the separate components of the multi-component vascular occlusion device herein downstream or distally within the lumen of the vessel 10. The presence of the expandable anchor component 100 can partially occlude the lumen of the vessel 10. For instance, as illustrated in FIG. 1A, a volume of blood flow (e.g., represented by the arrow identified by element number 115) that is proximal to or at a proximal end 114 of the expandable anchor component 100 can be less than a volume of blood flow (e.g., represented by the arrow identified by element number 117) that is distal to or at a distal end 116 of the expandable anchor component 100. That is, the primary function or purpose of the expandable anchor component 100 can be to provide a structural support which can anchor the expandable anchor component 100 (and subsequent component(s)) to the inner wall of the vessel 10.

In some embodiments, the expandable anchor component 100 may be repositionable and/or retractable. For instance, the expandable anchor component 100 can include a repositioning feature 111 configured to permit the expandable anchor component 100 to be repositionable from one location in a vessel to another location in a vessel and/or to be retracted from a target site in a vessel (e.g., entirely withdrawn from the vessel). For example, the repositioning feature 111 can be configured to radially contract (e.g., to a contracted configuration) at least a portion of the expandable anchor component 100 relative to an inner wall of the vessel to permit the expandable anchor component 100 to be repositioned within and/or retracted from the vessel. The repositioning feature 111 can be a wire, loop of material or other element that extends proximally from the body of the expandable anchor component 100. For instance, the repositioning feature 111 can be a wire that is coupled to and configured to hold intact at least two opposing couplers (not illustrated) which are clasped or otherwise configured to mechanically be held together. The two couplers may be configured to move to or be dispositioned at a radially expanded position at a site within a vasculature such as the target site. The at least two couplers may also be configured to move to a radially contracted position responsive to a proximal movement of the wire, thereby causing the at least two couplers to release from the site to permit the expandable anchor component 100 to be repositioned (e.g., moved proximally or distally) from the site.

The expandable anchor component 100 (e.g., individually or along with one or more other component of a multi-component vascular occlusion device 50) can be configured to be delivered to a target site via an insertion device such as a catheter. For instance, the expandable anchor component 100 can be configured to be delivered by a microcatheter, among other possibilities. The microcatheter can have a radial cross-section that is 3 FR or less. In some embodiments, the expandable anchor component 100 and an embolic component 130, as described herein, can be configured to be delivered together via the same catheter. For instance, the expandable anchor component 100 and embolic component 130 can be delivered as separate components via the same microcatheter. In some embodiments, the expandable anchor component 100, the embolic component 130, and the entrapment component, as described herein, can be configured to be delivered together via the same catheter.

FIG. 1B illustrates an example multi-component vascular occlusion device 50 comprising the expandable anchor component 100 and an embolic component 130 that may be delivered separately and is disposed within or integrated with the expandable anchor component 100 once delivered to the target site. The embolic component 130 may be substantially impermeable to fluid such that the embolic component 130 is configured to substantially occlude and/or stop fluid flow through the lumen of the vessel 10, either initially and/or over time. In some instances, vessel occlusion may take place slowly over time. As time passes following vessel occlusion, thrombus formation and/or tissue overgrowth on and/or around the embolic component 130 may take place, further occluding and/or sealing off the lumen of the vessel 10. In some instances, vessel occlusion may occur very quickly and/or immediately. Some suitable but non-limiting materials for the embolic component 130, for example metallic materials, polymer materials, composite materials, etc., are described below.

In some embodiments, at least a portion or all of the embolic component 130 can be disposed at least partially within the expandable anchor component 100. For instance, as illustrated in FIG. 1B, a portion of the embolic component 130 can be disposed within the expandable anchor component 100 and can contact at least a portion of the perimeter of the interior space 101 of the expandable anchor component 100. For instance, the embolic component 130 can occupy an entirety or majority of the interior space 101 of the expandable anchor component 100 to block blood flow through the vessel 10. For example, a portion of the embolic component 130 can be disposed between at least a portion of the body of the expandable anchor component 100 (e.g., between coils) to block blood flow through the vessel 10. Other configurations are also contemplated.

In some embodiments, the expandable anchoring component 100 may define a length measured along a central longitudinal axis of the multi-component occlusion device that is equal to or greater than a length of the embolic component 130.

FIGS. 2A-2C illustrate components of another example of a multi-component vascular occlusion device disposed in a lumen of a vessel. FIG. 2A is similar to FIG. 1A, but employs a different type of expandable anchor component 100. Namely, in FIG. 2A the expandable anchor component 100 is a coil, rather than the expandable stent or basket illustrated in FIG. 1A.

FIG. 2B illustrates the presence of an entrapment component 150. The entrapment component 150 is separate from the expandable anchor component 100 and the embolic component 130. The entrapment component 150 can be a screen (e.g., a polymeric and/or metallic fine mesh), coil, liquid, foam, gel, fiber, polymer, or any combination thereof, among other possibilities. For instance, the entrapment component 150 can be a circular mesh having a substantially uniform cross-section (e.g., a substantially uniform radial and/or longitudinal cross-section). The entrapment component 150 can be a screen or mesh that is configured with gaps or interstitial spaces therein which are sized and/or shaped to entrap at least some of the embolic component 130 therein, once the embolic component 130 is delivered to the target site. That is, a primary function of the entrapment component 150 can be to contact a proximal end or otherwise remain disposed within the expandable anchor component 100 and thereby subsequently trap at least a portion of the embolic component 130 within gaps or interstitial spaces in the entrapment component 150. For instance, the entrapment component 150 can be configured (e.g., sized and/or shaped) to friction fit at least partially within the perimeter of the interior space 101 disposed within the expandable anchor component 100 or can be configured to abut a proximal end of the expandable anchor component while the expandable anchor component exerts the radially outward force against the interior wall of the vessel in the expanded configuration. Thus, the entrapment component 150 may provide additional surface area for the embolic component 130 to contact at the target site.

As such, the presence of the entrapment component 150 can ensure that at least a majority of the embolic component 130 remains at the target site, thereby ensuring that that occlusion of the target site occurs once the multi-component vascular occlusion device is formed at the target site. For instance, the entrapment component 150 may be utilized in multi-component vascular occlusion device deployed in larger vessels (e.g., 6 mm or larger vessels) to ensure the occlusion of the target site occurs. The entrapment component 150 can be configured to be deployed within the lumen of the vessel 10 subsequent to deployment of the expandable anchor component 100 and prior to the embolic component 130 within the lumen of the vessel 10, as described herein. The presence of the entrapment component 150 can partially occlude the lumen of the vessel 10. For instance, as illustrated in FIG. 2B, a volume of blood flow (e.g., represented by the arrow identified by element number 119) that is distal to or at a distal end 116 of the expandable anchor component 100 can be further reduced as compared to a volume of blood flow (e.g., represented by the arrow identified by element number 117) when the expandable anchor component 100 is present.

FIG. 2C illustrates a multi-component vascular occlusion device 60 that is similar to the multi-component vascular occlusion device 50 in FIG. 1B, but includes the entrapment component 150 and illustrates the embolic component 130 being substantially disposed within and/or proximal to the entrapment component 150. As mentioned, the multi-component device including three components (the expandable anchor component 100, the entrapment component 150, and the embolic component 130) can be utilized in larger vessels (e.g., 6 mm or larger vessels) and/or higher blood flow volume vessels to ensure the occlusion of the target site occurs once the multi-component vascular occlusion device 60 is formed at the target site therein. The embolic component 130 in FIG. 2C can be the same type or a different type of embolic component 130 than the embolic component 130 in FIG. 1B. For instance, the expandable anchor component 100 can be a coil, and the embolic component 130 can be a liquid embolic material, as illustrated in FIG. 2C.

In some embodiments, a multi-component vascular occlusion device (e.g., multi-component vascular occlusion devices 50, 60) herein may have a maximum outer extent of about 8 mm (e.g., about 4 mm about a central longitudinal axis of the vascular occlusion device), and may be configured, designed, intended, and/or rated for use in a vessel 10 having an inner diameter of about 3-6 mm. For instance, the two component (expandable anchor component and the embolic component) multi-component vascular occlusion devices herein may be employed with a vessel having a vessel inner diameter of about 3-6 mm. However, the disclosure is not so limited and such two component multi-component vascular occlusion devices may be employed in vessels have a smaller or larger diameter. Other dimensions, positions, variations, and/or combinations are also contemplated.

In some embodiments, the multi-component vascular occlusion devices herein may have a maximum outer diameter of about 6 mm or about 8 mm (e.g., about 4 mm about a central longitudinal axis of the vascular occlusion device), and may be configured, designed, intended, and/or rated for use in a vessel 10 having a vessel inner diameter of greater than 6 mm. For instance, the three component multi-component vascular occlusion devices herein (e.g., including the expandable anchor component, the entrapment component, and the embolic component) may be employed with a vessel having a vessel inner diameter of greater than 6 mm. However, the disclosure is not so limited and the multi-component vascular occlusion devices may be employed in vessels with a different (e.g., smaller) diameter. Other dimensions, positions, variations, and/or combinations are also contemplated.

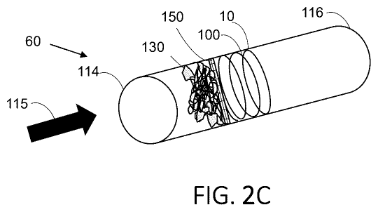

FIG. 3 illustrates an example of a kit 303 for forming the multi-component vascular occlusion device (e.g., the multi-component vascular occlusion device of FIG. 1B). As illustrated in FIG. 3, the kit 303 can include a plurality of separate components including an expandable anchor component 300 and an embolic component 330. Stated differently, the kit 303 can include at least the expandable anchor component 300 and the embolic component 330 which are not coupled together and are provided as separate components in the kit 303.

The expandable anchor component 300 and an embolic component 330 in the kit 303 can be configured to form a multi-component vascular occlusion device for deployment within a lumen of a vessel, as described herein. For instance, the expandable anchor component 300 and an embolic component 330 can be analogous or similar to the expandable anchor components 100 and the embolic component 130, described herein. The embolic component 330 can be a mechanical embolic component, a liquid embolic component, or a combination of a mechanical embolic component and a liquid embolic component, as described herein. The expandable anchor component 300 can be configured to be deployed within the lumen of the vessel and the embolic component 330 can be configured to be deployed within the lumen of the vessel subsequent to deployment of expandable anchor component 300 within the lumen of the vessel, as described herein.

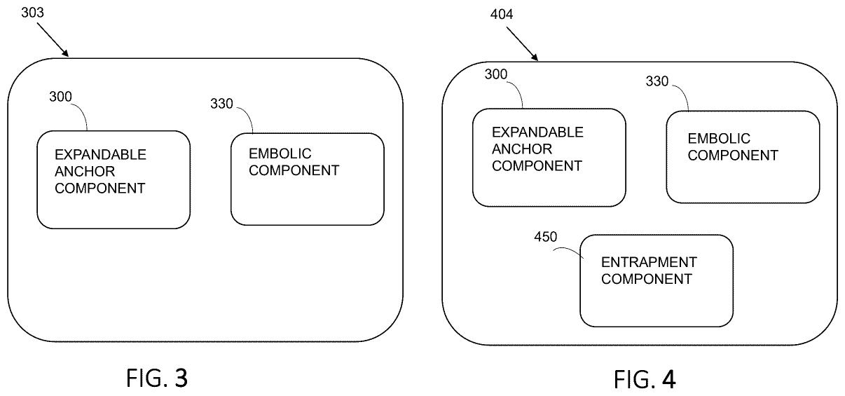

FIG. 4 illustrates an example of a kit 404 for forming the multi-component vascular occlusion device (e.g., the multi-component vascular occlusion device of FIG. 2C). The kit 404 is analogous to the kit 303, but additionally includes a separate entrapment component 450. The entrapment component 450 is separate from the expandable anchor component 400 and the embolic component 430. The entrapment component 450 can be analogous or similar to the entrapment component 150 described herein. The entrapment component 450 can be configured to be deployed within the lumen of the vessel subsequent to deployment of the expandable anchor component 400 and prior to deployment of the embolic component 430 within the lumen of the vessel, as described herein.

The components of the kits herein can be provided in any suitable type of container such as a sterile container. The components in the kits can be provided as sterilized components and/or may be suitable to undergo sterilization. The kits herein may include additional components (e.g., an insertion catheter, a push rod, etc.) not expressly described with respect to FIGS. 3-4.

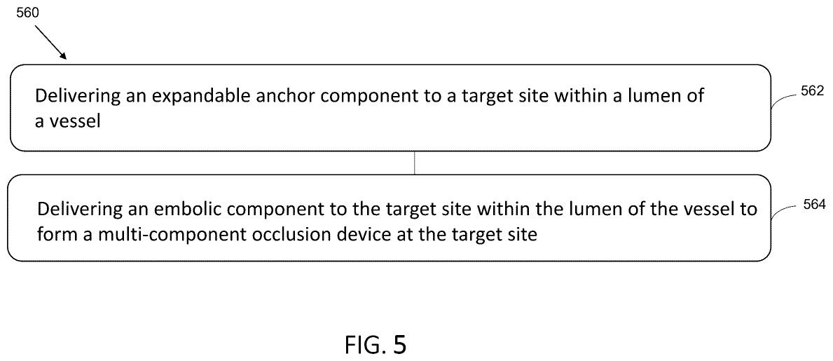

FIG. 5 illustrates an example of a method 560 of formation and/or use of a multi-component vascular occlusion device. For instance, at 562 the method 560 can include delivering an expandable anchor component, as described herein, to a target site within a lumen of a vessel. The expandable anchor component can be delivered initially (e.g., as a first component) of a multi-component vascular occlusion device that is formed at the target site. That is, the expandable anchor component can be delivered prior to any subsequent components (e.g., an embolic component, etc.) of the multi-component vascular occlusion device.

At 564, the method 560 can include delivering an embolic component, as described herein, to the target site within the lumen of the vessel. That is, the embolic component can be delivered to the target site (e.g., the same target site) subsequent to delivery of the expandable anchor component to form a multi-component vascular occlusion device at the target site. Once delivered to the target site, the embolic component can be disposed at least partially within the expandable anchor component to contact at least a portion of the perimeter of the interior space, as described herein, thereby forming a multi-component vascular occlusion device (e.g., including or only including the expandable anchor component and the embolic component). In some embodiments, the embolic component may be configured to not be disposed within the expandable anchor component when the expandable anchor component is initially in the collapsed configuration and may be configured to subsequently be disposed at least partially within the expandable anchor component while the expandable anchor component is in the expanded configuration. That is, the embolic component can be disposed within the expandable anchor component subsequent to delivery to the expandable anchor component to a target site. Similarly, in embodiments where an entrapment component is present, the embolic component can be disposed at least partially within the entrapment component subsequent to delivering the entrapment component to a target site, as detailed herein.

Disposing the embolic component at least partially within the expandable anchor component (e.g., within at least a portion of the interstitial spaces or gaps in a body of the expandable anchor component can occur due to a mechanical interaction (e.g., friction fit, etc.) between the expandable anchor component and the embolic component, once the embolic component is delivered to the target site. For example, the embolic component can be a mechanical embolic component (e.g., a mesh or filter) that is configured (e.g., sized and/or shaped) to contact the body of the expandable anchor component at the target site and/or which may be retained within the body of the expandable anchor component at least due to pressure imparted by blood flow on the mechanical embolic component. Similarly, in some embodiments the embolic component can be a chemical embolic component that is configured to contact the body of the expandable anchor component and be retained within the body of the expandable anchor component due to a chemical reaction or physical state change (e.g., a transition from an initial liquid state or gel state while the chemical embolic component is deployed to a solid state of the chemical embolic component at the target site). That is, in some embodiments the embolic component may comprise an embolic fluid such as an liquid embolic (e.g., an embolic glue), embolic foam, or an embolic gel. In some embodiments, the embolic fluid is dried and then granulated into particles of suitable size and is delivered as granulated particles. Granulating may be by any suitable process, for instance by grinding (including cryogrinding), homogenization, crushing, milling, pounding, or the like. Sieving or other known techniques can be used to classify and fractionate the particles.

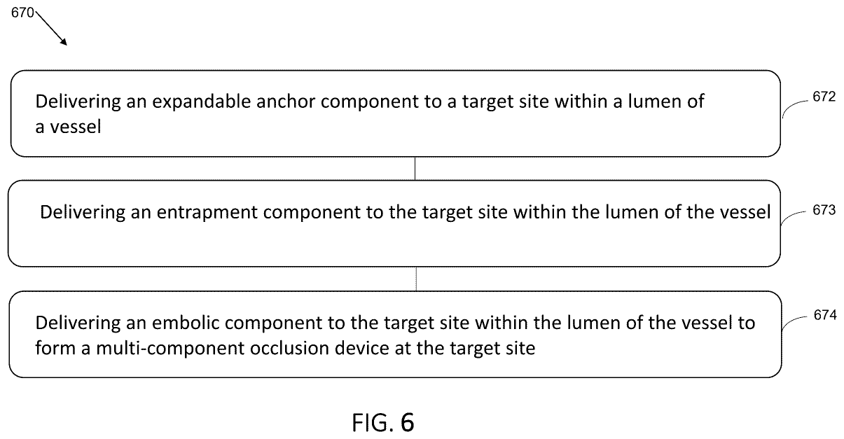

In some embodiments, the methods can include additional elements such as repositioning the multi-component vascular occlusion device at a target site, retracting the multi-component vascular occlusion device from a target site, dilation of a target site, imaging a target site, and/or delivery of additional components or equipment (e.g., imaging equipment) to a target site. For example, FIG. 6 illustrates an example of another method 670 of use and/or forming a multi-component vascular occlusion device. The method 670 is analogous to the method 560, with the addition of delivery of an entrapment component, as described herein, to a target site.

For instance, at 672, the method 670 can include delivering an expandable anchor component to a target site within a lumen of a vessel, as described herein. At 673, the method 670 can include delivering an entrapment component to the target site (e.g., the same target site). As mentioned, the entrapment component is separate from the expandable anchor component and the embolic component, and thus can be delivered separately to the target site. For example, the method 670 can include delivering the entrapment component to the target site after delivery of the expandable anchor component to the target site and prior to delivering the embolic component to the target site. At 674, the method 670 can include delivering an embolic component to the target site to form a multi-component vascular occlusion device (e.g., including or only including three components in the form of the expandable anchor component, the entrapment component and the embolic component). That is, the embolic component can be delivered to the target site after each of the expandable anchor component and the entrapment component to form the multi-component vascular occlusion device.

The methods described herein (e.g., described with respect to FIGS. 5-6) can be performed with the individual components and/or the multi-component vascular occlusion devices described herein. Delivery of the components of the multi-component vascular occlusion devices (e.g., sequential delivery of the individual components) described herein can be performed via one or more catheters. For instance, in some embodiments each of the components in the methods 560/670 can be delivered via an individual catheter, for instance, by retaining the individual catheter at or proximal to the target site and delivering respective components via the catheter while the catheter remains at or proximal to the target site. That is, an individual catheter may be navigated proximal to or at a location of a target site, the expandable anchor component may be delivered via the individual catheter to the target site, and at least the embolic component may be delivered via the individual catheter to the target site. For instance, in some embodiments, at least the expandable anchor component and the embolic component are configured to be deployed via the same individual catheter at different times to a target site such as the same individual microcatheter. Similarly, in some embodiments each of the expandable anchor component, the entrapment component, and the embolic component are configured to be deployed via the same individual catheter such as the same individual microcatheter.

The materials that can be used for the various components of the multi-component vascular occlusion devices herein may include those commonly associated with medical devices. For instance, in some embodiments, one or more components of the multi-component vascular occlusion devices herein, and/or portions thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material. Additional examples of suitable materials or components (e.g., for repositioning features, coils, and/or embolic fluids herein, etc.) include those commercially available as Embold™ and the like (available from Boston Scientific Corporation), Interlock™ and the like (available from Boston Scientific Corporation), Ruby™ (available from Penumbra, Inc.), Concerto™ and the like (available from Medtronic, Inc.), Azur CX™ and the like (available from Terumo corporation), Prestige (Balt) and the like, various materials (e.g., liquids, gels, glues) such as Obsidio™ and the like (available from Boston Scientific Corporation), Lava™ and the like (available from SirTex Medical Inc), Onyx™ and the like (available from Medtronic, Inc.), Contour™ and the like (available from Boston Scientific Corporation), LC Bead™ and the like (available from Boston Scientific Corporation), and Embocube™ and the like (available from Merit Medical Systems, Inc.)

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, one or more components of the multi-component vascular occlusion devices herein or portions thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the one or more components of the multi-component vascular occlusion devices herein, etc. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of one or more components of the multi-component vascular occlusion devices herein or portions thereof, etc. to achieve the same result.

In some embodiments, a degree of Magnetic Resonance Imaging (MRI) compatibility is imparted into one or more components of the multi-component vascular occlusion devices herein. For example, one or more components of the multi-component vascular occlusion devices herein and/or portions thereof, may be made of a material that does not substantially distort the image and create substantial artifacts (e.g., gaps in the image). Certain ferromagnetic materials, for example, may not be suitable because they may create artifacts in an MRI image. One or more components of the multi-component vascular occlusion devices herein or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, one or more components of the multi-component vascular occlusion devices herein and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, one or more components of the multi-component vascular occlusion devices herein and/or portions thereof, may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, one or more components of the multi-component vascular occlusion devices herein and/or portions thereof, may include and/or be formed from a textile material. Some examples of suitable textile materials may include synthetic yarns that may be flat, shaped, twisted, textured, pre-shrunk or un-shrunk. Synthetic biocompatible yarns suitable for use in the present invention include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun-types. The type and denier of the yarn chosen may be selected in a manner which forms a biocompatible and implantable prosthesis and, more particularly, a vascular structure having desirable properties.

In some embodiments, one or more components of the multi-component vascular occlusion devices herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A multi-component vascular occlusion device for deployment within a lumen of a vessel, the multi-component vascular occlusion device comprising:
an expandable anchor component configured to shift between a collapsed configuration and an expanded configuration, wherein the expandable anchor component is configured in the expanded configuration to exert a radially outward force against an interior wall of the vessel; and
an embolic component separate from and disposed at least partially within the expandable anchor component.

2. The multi-component vascular occlusion device of claim 1, wherein the embolic component is configured to not be disposed within the expandable anchor component when the expandable anchor component is initially in the collapsed configuration and subsequently be disposed at least partially within the expandable anchor component while the expandable anchor component is subsequently in the expanded configuration.

3. The multi-component vascular occlusion device of claim 1, wherein the expandable anchor component comprises a frame including a wire, a mesh, or a screen.

4. The multi-component vascular occlusion device of claim 1, wherein the expandable anchor component comprises a coil.

5. The multi-component vascular occlusion device of claim 1, wherein the embolic component is a mechanical embolic component, the mechanical embolic component including a wire, a mesh, or a screen.

6. The multi-component vascular occlusion device of claim 1, wherein the embolic component comprises an embolic fluid, the embolic fluid comprising a embolic liquid, embolic foam, or an embolic gel.

7. The multi-component vascular occlusion device of claim 1, wherein the expandable anchor component is configured to be deployed within the lumen of the vessel, and wherein the embolic component is configured to be deployed within the lumen of the vessel subsequent to deployment of the expandable anchor component within the lumen of the vessel.

8. The multi-component vascular occlusion device of claim 1, further comprising an entrapment component that is separate from the expandable anchor component and the embolic component.

9. The multi-component vascular occlusion device of claim 8, wherein the entrapment component is configured to be deployed within the lumen of the vessel subsequent to deployment of the expandable anchor component and prior to the embolic component within the lumen of the vessel.

10. The multi-component vascular occlusion device of claim 8, wherein the expandable anchor component defines a perimeter of an interior space disposed within the expandable anchor component and the entrapment component is configured to:
friction fit at least partially within the perimeter of the interior space disposed within the expandable anchor component; or
abut a proximal end of the expandable anchor component while the expandable anchor component exerts the radially outward force against the interior wall of the vessel in the expanded configuration.

11. The multi-component vascular occlusion device of claim 8, wherein the entrapment component includes a mesh, a coil, a liquid, a foam, a gel, or any combination thereof.

12. The multi-component vascular occlusion device of claim 11, wherein the expandable anchor component is a coil.

13. The multi-component vascular occlusion device of claim 12, wherein the embolic component is a liquid embolic material.

14. The multi-component vascular occlusion device of claim 1, wherein the expandable anchor component includes a repositioning feature.

15. The multi-component vascular occlusion device of claim 1, wherein at least the expandable anchor component and the embolic component are configured to be deployed via the same individual microcatheter.

16. A multi-component vascular occlusion device for deployment within a lumen of a vessel, the multi-component vascular occlusion device comprising:
an expandable anchor component configured to shift between a collapsed configuration and an expanded configuration, the expandable anchor component defining a perimeter of an interior space disposed within the expandable anchor component, wherein the expandable anchor component is configured in the expanded configuration to exert a radially outward force against an interior wall of the vessel;
an entrapment component separate from the expandable anchor component and including a mesh that is friction fit at least partially within the perimeter of the interior space disposed within the expandable anchor component or abuts a proximal end of the expandable anchor component; and
an embolic component separate from the expandable anchor component and the entrapment component, wherein the embolic component is disposed i) at least partially within the mesh and ii) at least partially within the expandable anchor component to contact at least a portion of the perimeter of the interior space.

17. The multi-component vascular occlusion device of claim 16, wherein the vessel has an inner diameter of 6 millimeters or greater, and wherein the expandable anchor component is a coil configured to exert the radially outward force against the interior wall of the vessel in the expanded configuration that is sufficient to retain the multi-component vascular occlusion device in the vessel.

18. A kit for forming a multi-component vascular occlusion device for deployment within a lumen of a vessel, the kit comprising:
an expandable anchor component configured to shift between a collapsed configuration and an expanded configuration, the expandable anchor component, wherein the expandable anchor component is configured to exert a radially outward force against an interior wall of the vessel in the expanded configuration; and
an embolic component separate from and configured to be disposed at least partially within the expandable anchor component while the expandable anchor component exerts the radially outward force against the interior wall of the vessel to form the multi-component vascular occlusion device.

19. The kit of claim 18, wherein the expandable anchor component and the embolic component are separate elements.

20. The kit of claim 19, wherein the kit further comprises an entrapment component, and wherein the entrapment component is separate from the expandable anchor component and the embolic component.
